(19) United States
(12) Patent Application Publication
(10) Pub. No.: US 2023/0352187 A1
(43) Pub. Date: Nov. 2, 2023

(54) APPROACHES TO LEARNING, DOCUMENTING, AND SURFACING MISSED DIAGNOSTIC INSIGHTS ON A PER-PATIENT BASIS IN AN AUTOMATED MANNER AND ASSOCIATED SYSTEMS

(71) Applicant: Magenta Care Continuum Inc., Denver, CO (US)

(72) Inventors: Justin D. Schaper, Plano, TX (US); Nancy S. Stimson, Golden, CO (US); George F. Adam, Jr., Denver, CO (US)

(21) Appl. No.: 18/308,128

(22) Filed: Apr. 27, 2023

Related U.S. Application Data
(60) Provisional application No. 63/335,498, filed on Apr. 27, 2022.

(51) Int. Cl. G16H 50/70; G16H 10/60

(52) U.S. Cl. CPC G16H 50/70; G16H 10/60

(57) ABSTRACT

Introduced here is a computer-aided diagnostic system that is able to surface diagnostic insights through analysis of data related to claims submitted to insurers for reimbursement purposes. At a high level, the system enables diagnoses that should be associated with individual patients to be more reliably detected, lessening the likelihood of negative diagnoses being false negatives. The system is distinguishable from conventional diagnostic support systems in that the analysis is retrospective, using claims-related datasets. Note that, in some embodiments, the system may examiner, consider, or otherwise incorporate clinical data, though its analysis is primarily focused on claims datasets. Through the retrospective lens, patterns of diagnoses, treatments, and consultations can be surfaced by the system. These patterns can be helpful in suggesting diseases that are not readily apparent in clinical data that is available at the time of treatment.

### CROSS-REFERENCE TO RELATED APPLICATIONS

This application claims priority to U.S. Provisional Application No. 63/335,498, titled “Approaches to Surfacing Missed Diagnostic Groupings on a Per-Patient Basis in an Automated Manner and Associated Systems” and filed on Apr. 27, 2022, which is incorporated by reference herein in its entirety.

### TECHNICAL FIELD

Various embodiments concern computer programs and associated computer-implemented techniques for surfacing insights into health through automated, programmatic analysis of claims submitted for reimbursement purposes.

### BACKGROUND

In the United States healthcare system, the term “medical billing” is commonly used to refer to a process in which a healthcare provider (or simply “provider”) obtains insurance information from a patient and then files a claim with an insurer in order to receive payment for services rendered. The process may be comparable regardless of whether those services relate to low-cost testing procedures—like blood draws and biopsies—or high-cost operating procedures. Moreover, the same process is generally used for most insurers, whether private companies or government-sponsored programs, namely, coding reports are compiled to indicate diagnoses made and procedures performed and then prices are applied accordingly.

The interaction generally begins with a visit to a healthcare facility—a healthcare professional (or simply “professional”) will typically meet with a patient and then create or update her health record as necessary. Then, diagnosis and procedure codes (or simply “codes”) are normally assigned to the visit (and therefore, the patient) by a clinical coder. Clinical coders (also called “diagnostic coders” or “medical coders”) are professionals—usually employed by providers—whose main duties are to analyze clinical statements included in the health record and assign codes using a classification scheme. These codes assist insurers in determining coverage and medical necessity of the services rendered by providers to patients. Accordingly, the classification scheme can be used to transform descriptions of diagnoses and procedures into standardized statistical codes.

The classification scheme may include a list of diagnostic codes that are associated with different diseases, ailments, and conditions. Moreover, the classification scheme may include a list of procedure codes that can be used to capture interventional information. Together, the diagnosis and procedure codes are used by providers to document the care provided to patients, as well as seek reimbursement for the care. Claims are usually electronically formatted as American National Standards Institute (“ANSI”) 837 files and submitted using the Electronic Data Interchange, so as to ensure some degree of consistency across different providers and insurers.

### BRIEF DESCRIPTION OF THE DRAWINGS

FIG. 1 illustrates a network environment that includes a diagnostic system that is executed by a computing device.

FIG. 2 illustrates an example of a computing device that is able to implement a diagnostic system designed to surface diagnostically relevant insights through analysis of claims datasets associated with patients.

FIG. 3 illustrates how data can be processed as it flows through a diagnostic system.

FIG. 4 includes an example of a suspect list that may be produced by the diagnostic system as output.

FIG. 5 includes a high-level illustration of an exemplary architecture of a diagnostic system.

FIG. 6 depicts an example of a database entity relationship diagram.

FIG. 7 is a block diagram illustrating an example of a processing system in which at least some operations described herein can be implemented.

Various features of the technology described herein will become more apparent to those skilled in the art from a study of the Detailed Description in conjunction with the drawings. Various embodiments are depicted for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

Several entities have developed computer-aided diagnostic support systems in an effort to facilitate the process by which patients are diagnosed and those diagnoses are documented. As an example, imaging has historically been an effective means for detecting cancer, and diagnostic support systems have become a key part of identifying the pathologically relevant features in digital images that are indicative of cancer. As another example, diagnostic support systems have been developed and then trained to identify indicators of cardiopulmonary disease through analysis of echocardiograms and corresponding reports that summarize the findings of professionals.

Diagnostic support systems have proven useful in detecting a variety of different ailments. However, the analysis performed by these diagnostic support systems is highly dependent on the quality and quantity of information that is available for patients of interest. Simply put, if sufficient information—regardless of form—is not available for analysis, a diagnostic support system may not be able to determine whether to predict a positive diagnosis or a negative diagnosis. The term “positive diagnosis” may be used to refer to a scenario where a patient is determined to have a given disease, while the term “negative diagnosis” may be used to refer to a scenario where a patient is determined to not have a given disease.

Moreover, fully relying on patient-specific information to predict diagnoses may result in patients being improperly diagnosed. Assume, for example, that a diagnostic support system predicts a negative diagnosis for a patient based on an analysis of her information. There is a chance—especially if the amount of information that is available to the diagnostic support system is small—that the negative diagnosis is representative of a false negative. The term “false negative” refers to a prediction that indicates the patient does not have the disease when the patient actually does have the disease. Because the prediction is based entirely on the information provided to the diagnostic support system as input, there are few options for reliably lowering the likelihood of false negatives.

Introduced here is a computer-aided diagnostic system (or simply “system”) that is able to surface diagnostic insights through analysis of data related to claims submitted to insurers for reimbursement purposes. At a high level, the system enables diagnoses that should be associated with individual patients to be more reliably detected, lessening the likelihood of negative diagnoses being false negatives. The system is distinguishable from conventional diagnostic support systems in that the analysis is retrospective, using claims-related datasets (also called “claims datasets”) as further discussed below. Note that, in some embodiments, the system may examiner, consider, or otherwise incorporate clinical data, though its analysis is primarily focused on claims datasets. Through the retrospective lens, patterns of diagnoses, treatments, and consultations can be surfaced by the system. These patterns can be helpful in suggesting diseases that are not readily apparent in clinical data that is available at the time of treatment. Simply put, the system can surface insights into health that simply are not discoverable by professionals, as those professionals do not have access to comprehensive claims datasets (and would not be able to draw general conclusions regarding patterns of diagnoses, treatments, and consultations even if such claims datasets were available).

By detecting these missing diagnoses, the system helps to close “gaps” in healthcare by enabling better treatment. Further, the system can help determine patterns of best practice (e.g., on a per-disease, per-provider, or per-professional basis), and the system can ensure that providers are appropriately reimbursed based on the actual health states of patients. As a starting point, the system may use claims datasets that are sourced directly from providers or insurers. Such an approach allows the system to detect smaller scale patterns that may be indetectable in larger datasets. For example, the system may be able to detect regional differences in how diagnoses are assigned (e.g., by different professionals or at facilities associated with different providers), while also generating more generally applicable rules. Specifically, the system may develop “local rules” that are suitable for specific professionals or providers and “national rules” that are suitable for all professionals and providers for which claims datasets are available.

For the purpose of illustration, embodiments may be described with reference to certain system architectures. However, those skilled in the art will recognize that the features of those embodiments may be similarly applicable to other system architectures. For example, while the system may be described as being implemented using a cloud computing service, those skilled in the art will recognize that the system could be implemented as a standalone computer program.

Moreover, embodiments may be described in the context of executable instructions for the purpose of illustration. However, those skilled in the art will recognize that the system could be implemented via hardware or firmware instead of, or in addition to, software. As an example, a computer program that is able to perform aspects of the approaches described herein may be executed by the processor of a computing device. This computer program may interface—directly or indirectly—with hardware, firmware, or other software implemented on the computing device or another computing device that is communicatively accessible. For instance, the computer program may access a datastore maintained on another computing device in order to obtain a claims dataset for training purposes or obtain a claims dataset for inferencing purposes.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

The term “based on” is to be construed in an inclusive sense rather than an exclusive sense. That is, in the sense of “including but not limited to.” Thus, unless otherwise noted, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the word “or” is intended to cover all of the following interpretations: any of the items in the list, all of the items in the list, and any combination of items in the list.

### Overview of Diagnostic System

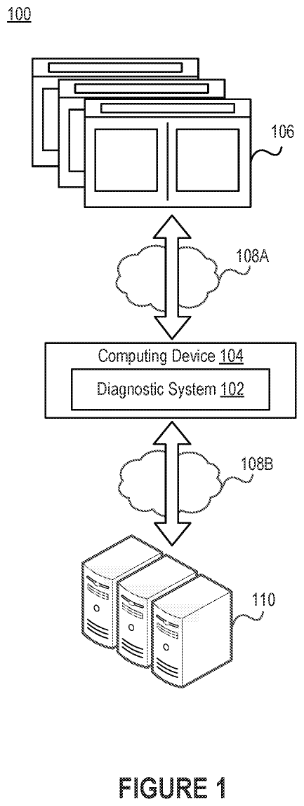

FIG. 1 illustrates a network environment 100 that includes a diagnostic system 102 that is executed by a computing device 104. An individual (also called a “user”) may be able to interact with the diagnostic system 102 via interfaces 106. For example, a user may be able to access an interface through which to select a claims dataset for which a rule set is to be generated as part of a training process. As another example, a user may be able to access an interface through which a claims dataset can be selected, such that the rule set can be applied thereto to produce an output as part of an inferencing process. Moreover, the user may be able to review outputs produced by the rule set upon being applied to the claims dataset. Interfaces may be configured to “guide” users through the training and inferencing processing, so that even individuals without expertise in developing or applying learned rules—like professionals, such as physicians and nurses—can use the diagnostic system 102. Some interfaces are configured to facilitate interactions between patients and professionals, while other interfaces are configured to serve as informative dashboards for either patients or professionals.

As shown in FIG. 1, the diagnostic system 102 may reside in a network environment 100. Thus, the computing device 104 on which the diagnostic system 102 resides may be connected to one or more networks 108A-B. Depending on its nature, the computing device 104 could be connected to a personal area network (“PAN”), local area network (“LAN”), wide area network (“WAN”), metropolitan area network (“MAN”), or cellular network. For example, if the computing device 104 is a computer server, then the computing device 104 may be accessible to users via respective computing devices that are connected to the Internet via LANs.

The interfaces 106 may be accessible via a web browser, desktop application, mobile application, or another form of computer program. For example, to interact with the diagnostic system 102, a user may initiate a web browser on the computing device 104 and then navigate to a web address associated with the diagnostic system 102. As another example, a user may access, via a mobile application executing on her mobile phone or a desktop application executing on her laptop computer, interfaces that are generated by the diagnostic system 102 through which she can select claims datasets for training purposes, select claims datasets for inferencing purposes, review analyses of claims datasets, and the like. Diagnostically relevant information could also be provided through the interfaces. Such information can include patient information, such as patient name, date of birth, disease history, symptoms, medications, treatments, or provider information, such as professional name, facility name, or geographical location. Alternatively, diagnostically relevant information may be automatically populated into the interface by the diagnostic system 102 (e.g., in response to discovering such information in metadata that accompanies the claims dataset or corresponding clinical dataset or extracting such information from either of those datasets). As further discussed below, the diagnostically relevant information may be used by the diagnostic system 102 to establish which rules or rule sets should be applied to each claims dataset to surface meaningful insights.

The diagnostic system 102 may guide a user through a detection procedure in which a claims dataset associated with a patient is examined, through the application of one or more rules, to establish whether any possible diagnoses have been overlooked. The detection procedure may be representative of a step of the inferencing process, and therefore the terms “detection procedure” and “inferencing process” may be used interchangeably. To initiate the detection procedure, a user may access an interface through which to select the patient, a group of patients, or the claims dataset. The interfaces 106 are not necessarily limited to selecting claims datasets—and reviewing analyses of claims datasets—associated with patients, however. Through the interfaces 106, one or more claims datasets could be selected by a user. Then, the user may be able to review the rule set determined for (e.g., learned from) those claims datasets by the diagnostic system 102. The user may be permitted to alter the rule set, for example, by manually adding new rules, deleting existing rules, or modifying existing rules. Professionals may also be able to access the interfaces 106 in order to input or review information regarding disease status, progression, treatment, and the like. As an example, a claims dataset associated with a patient who has undergone examination and corresponding outputs produced by the diagnostic system 102 could be examined by a professional, who may be able to annotate the claims dataset, annotate a corresponding clinical dataset, or input notes (e.g., regarding diagnoses or treatment options). Accordingly, the rule set could be learned by the diagnostic system 102 in an entirely automated manner, or the rule set could be learned by the diagnostic system 102 in a semi-automated manner where modifications of individual rules are permitted. Interfaces 106 that are generated by the diagnostic system 102 may be accessible via various computing devices, including mobile phones, tablet computers, desktop computers, mobile workstations (also referred to as “medical carts”), and the like.

Generally, the diagnostic system 102 is executed by a cloud computing service operated by, for example, Amazon Web Services®, Google Cloud Platform™, or Microsoft Azure®. Thus, the computing device 104 may be representative of a computer server that is part of a server system 110. Often, the server system 110 is comprised of multiple computer servers. These computer servers can include rule sets, algorithms (e.g., for generating rules, for processing claims datasets, for processing clinical datasets), patient information (e.g., age, date of birth, disease classification, disease state, etc.), provider information (e.g., geographical location, names of employed professionals, services offered, etc.), and other assets. Those skilled in the art will recognize that this information could also be distributed amongst the network-accessible server system 110 and one or more computing devices, or maintained in a decentralized network infrastructure such as a blockchain.

As mentioned above, aspects of the diagnostic system 102 could be hosted locally, for example, in the form of a computer program executing on the computing device 104 that is accessible to a user. Several different versions of computer programs may be available depending on intended use. Assume, for example, that a user would like to complete a detection procedure in which a claims dataset is analyzed to determine the likelihood of a missed diagnosis. In such a scenario, the computer program may cause display of a series of interfaces that are intended to guide the user through the detection procedure. Note that in embodiments where the diagnostic system 102 is implemented, at least partially, on the computing device 104 that is accessible to the user, the diagnostic system 102 may still be communicatively connected to the network-accessible server system 110.

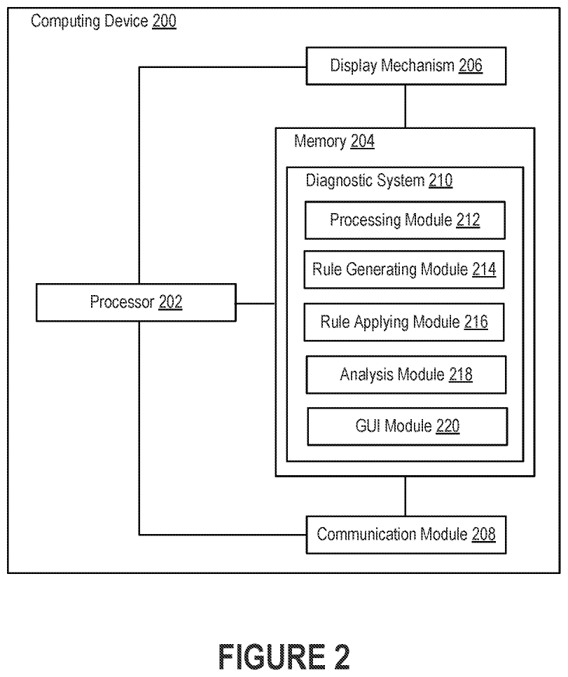

FIG. 2 illustrates an example of a computing device 200 that is able to implement a diagnostic system 210 designed to surface diagnostically relevant insights through analysis of claims datasets associated with patients. By examining these claims datasets through a retrospective lens, the diagnostic system 210 is able to discover and then address gaps in treatment or documentation that have been overlooked by professionals. As further discussed below, the diagnostic system 210 can apply rule sets or individual rules to these claims datasets to identify pieces of information that indicate these patients may have been misdiagnosed or mis-documented. These pieces of information may be referred to as “characteristics” or “features” of these patients.

As an example, assume that a physician accesses the computing device 200 to review a generated output that indicates the diagnostic system 210 has identified a potentially undiagnosed condition for a given patient. The physician can consider this information in her next examination of the patient to determine if evidence of the undiagnosed condition is clinically present, and therefore whether a diagnosis should be rendered for the undiagnosed condition. An additional example is where a medical coder performing billing operations accesses the computing device 200 to review generated output that indicates the diagnosis system 210 has identified a potentially undocumented condition for a given patient. The medical coder can review additional clinical documentation external to the diagnostic system 210 and determine whether the specific diagnosis coding is warranted but had not previously been filed in a claim. In this scenario, the medical coder may submit a correction to the responsible claim payer—whether an insurer or the patient herself.

As shown in FIG. 2, the computing device 200 can include a processor 202, memory 204, display mechanism 206, and communication module 208. Each of these components is discussed in greater detail below. Those skilled in the art will recognize that different combinations of these components may be present depending on the nature of the computing device 200. For example, if the computing device 200 is a computer server that is part of a server system (e.g., server system 110 of FIG. 1), then the computing device 200 may not include the display mechanism 206, though the computing device 200 may be communicatively connected to another computing device that does include a display mechanism.

The processor 202 can have generic characteristics similar to general-purpose processors, or the processor 202 may be an application-specific integrated circuit (“ASIC”) that provides control functions to the computing device 200. The processor 202 can be connected to all components of the computing device 200, either directly or indirectly, for communication purposes.

The memory 204 may be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 202, the memory 204 can also store data generated by the processor 202 (e.g., when executing the modules of the diagnostic system 210). Note that the memory 204 is merely an abstract representation of a storage environment. The memory 204 could be comprised of actual integrated circuits (also called “chips”).

The display mechanism 206 can be any mechanism that is operable to visually convey information to a user. For example, the display mechanism 206 may be a panel that includes light-emitting diodes (“LEDs”), organic LEDs, liquid crystal elements, or electrophoretic elements. In some embodiments, the display mechanism 206 is touch sensitive. Thus, the user may be able to provide input to the diagnostic system 210 by interacting with the display mechanism 206. Alternatively, the user may be able to provide input to the diagnostic system 210 through some other control mechanism.

The communication module 208 may be responsible for managing communications external to the computing device 200. The communication module 208 may be wireless communication circuitry that is able to establish wireless communication channels with other computing devices. Examples of wireless communication circuitry include 2.4 gigahertz (“GHz”) and 5 GHz chipsets compatible with Institute of Electrical and Electronics Engineers (“IEEE”) 802.11. Such chipsets are commonly called “Wi-Fi chipsets.” Alternatively, the communication module 208 may be representative of a chipset configured for Bluetooth®, Near Field Communication (NFC), and or another short-range communication protocol. Some computing devices—like mobile phones, tablet computers, and the like—are able to wirelessly communicate via separate channels. Accordingly, the communication module 208 may be one of multiple communication modules implemented in the computing device 200.

For convenience, the diagnostic system 210 may be referred to as a computer program that resides in the memory 204. However, the diagnostic system 210 could be comprised of hardware or firmware in addition to, or instead of, software. In accordance with embodiments described herein, the diagnostic system 210 may include a processing module 212, rule generating module 214, rule applying module 216, analysis module 218, and graphical user interface (“GUI”) module 220. These modules can be an integral part of the diagnostic system 210. Alternatively, these modules can be logically separate from the diagnostic system 210 but operate “alongside” it. Together, these modules may enable the diagnostic system 210 to develop and then implement rules to identify information suggestive of diseases that are not readily apparent in clinical datasets.

The processing module 212 can process data that is obtained by the diagnostic system 210 into a format that is suitable for the other modules. The nature of the processing may depend on whether the data obtained by the diagnostic system 210 is to be used for training purposes, inferencing purposes, or other purposes.

Assume, for example, that the diagnostic system 210 acquires multiple claims datasets to be used to generate rules that indicate how patients with different diseases were generally treated so as to achieve improved outcomes. These claims datasets could be obtained from providers, insurers, or both. The processing module 212 may “clean” these claims datasets in order to produce a curated dataset (also called a “training dataset”), for example, that includes several hundred examples of treatment per disease. Note that the size the training dataset may vary, as the number of diseases for which rules are to be generated may vary. However, the training dataset should generally include at least a predetermined number (e.g., 30, 50, 200, 500, or 1,000) of “samples” for each disease to ensure that the rules are generally applicable (and therefore, not generated for unusual scenarios rather than normal scenarios). In contrast to conventional machine learning algorithms, the approach described herein generally performs well even if the training dataset includes relatively few samples (e.g., several dozen or fewer). In general, low sample datasets are accessible under this approach and result in fewer false positive signals.

The claims datasets that are used by the diagnostic system 210 for training are generally representative of longitudinal data series that include information regarding the corresponding patients over extended intervals of time (e.g., months or years). As such, these claims datasets can serve as a reference—for the diagnostic system 210—as to which treatments were successful. This makes claims datasets useful for not only learning information that is suggestive of disease, but also learning information about which treatments are most appropriate—either based on success in treating or managing disease across a wide spectrum of patients or based on success in treating or managing disease across patients that share a characteristic (e.g., gender, age, ethnicity) with a patient for whom a proposed diagnosis is to be predicted.

Alternatively, the diagnostic system 210 could acquire a claims dataset to which a rule set is to be applied for inferencing purposes. Assume, for example, that the diagnostic system 210 acquires a claims dataset associated with a patient for whom a proposed diagnosis—either negative or positive—is available. The proposed diagnosis may have been determined by a professional, for example, while the claims dataset may have been generated by other providers treating the patient. In such a scenario, the processing module 212 may be responsible for applying operations to the claims dataset. Thus, the processing module 212 may process (e.g., filter, sample, or otherwise alter) the claims dataset acquired by the diagnostic system 210 to ensure that the claims dataset is usable by the other modules of the diagnostic system 210.

The rule generating module 214 may be responsible for implementing logic that calculates correlation probabilities between different datapoints in a claims dataset with the targeted diagnostic grouping for a population under study. Assume, for example, that the diagnostic system 210 obtains, as input, claims datasets that include information regarding patients known to have a given disease. Through analysis of the claims datasets, the rule generating module 214 can establish how different treatments or combinations of treatments correspond to outcome. For simplicity, the term “treatment combination” may be used to refer to singular treatments and combinations of multiple treatments. Each treatment combination may be associated with a measure that is representative of correlation with a positive diagnosis of the disease. For example, the rule generating module 214 may compute, derive, or otherwise establish, for each treatment combination, a correlation probability (also called a “probability metric” or “probability score”), and rules can be generated for those treatment combinations for which the probability score exceeds a threshold. At a high level, each rule may be representative of a data structure that specifies a treatment combination (e.g., using one or more treatment codes) that has been determined to sufficiently correlate to disease state. Accordingly, rules can be generated to catalogue the treatment combinations that are deemed to be successful in helping patients achieve positive outcomes.

Note that while the process for generating rules is described in the context of treatment combinations, a similar process could be carried out to generate rules based on other information that can be derived from claims datasets or clinical datasets. For example, through analysis of the claims datasets, the rule generating module 214 may establish how different patient characteristics correspond to outcome. Examples of patient characteristics include gender, age, ethnicity, smoking history, alcohol history, family history of disease, and the like. As mentioned above, each patient characteristic may be associated with a measure that is representative of correlation with a positive diagnosis of the disease, and the rule generating module 214 may generate a rule for each patient characteristic for which the measure exceeds a threshold. At a high level, these rules catalogue the patient characteristics that are determined to sufficiently correlate to disease state.

Moreover, the rule generating module 214 may compile rules into sets. A set could be generated for a given disease, or a set could be generated for a given professional or provider. Sets could also be generated for combinations of diseases, professionals, and providers. For example, a set could be generated for a given disease and professional, or a set could be generated for a given disease and provider. These rule sets may be referred to as “local rule sets” or “local sets,” as the rules should be applied in a more targeted manner. Additionally or alternatively, all rules that are generated by the rule generating module 214 may be compiled into a single set. This rule set may be referred to as the “global rule set” or “global set,” as the rules can be applied more liberally to claims data without regard for disease, professional, provider, etc.

The rule applying module 216 may be responsible for applying the rules generated by the rule generating module 214. Assume, for example, that the diagnostic system 210 obtains, as input, a claims dataset associated with a patient for whom additional analysis is desired. In such a scenario, the rule applying module 216 may apply at least one rule set to the claims dataset, so as to produce one or more outputs indicating whether further consideration by a professional is warranted. For example, the rule applying module 216 may apply multiple local rule sets (e.g., one associated with the professional responsible for treating the patient, one associated with a disease for which the patient was negatively diagnosed, etc.). As another example, the rule applying module 216 may apply the global rule set maintained in the memory 204. Generally, each rule applied to the claims dataset is designed to produce a separate output. Accordingly, the rule applying module 216 may produce a series of outputs by applying a rule set to the claims dataset, and each output included in the series of outputs may be representative of an indication as to whether a corresponding rule was determined to be a “match.”

The analysis module 218 may be responsible for examining the outputs produced by the rule sets applied by the rule applying module 216. In some scenarios, the outputs produced by the rule sets may not be particularly useful on their own. As such, the analysis module 218 may be responsible for considering the context of these outputs in a more holistic sense. For example, the analysis module 218 may consider outputs produced by the rule applying module 216, as well as other information related to the patient, to determine, derive, or infer an insight into the health of the patient. This other information could be input by the patient, input by a professional, extracted from a clinical dataset associated with the patient, derived from an electronic health record associated with the patient, etc. Generally, the analysis module 218 determines the appropriate course of action based on which rules, if any, were determined to “match” upon being applied to the claims dataset by the rule applying module 216. For example, if the analysis module 218 determines that a “matching rule” indicates there is a likelihood that the patient has a given disease, then the analysis module 218 may cause a notification to be generated and then presented to the patient or a professional. The notification may specify the likelihood, or the notification may simply indicate that further examination with respect to the given disease is recommended.

The GUI module 220 may be responsible for generating interfaces that can be presented on the display mechanism 206. Various types of information can be presented on these interfaces. For example, information that is derived, inferred, or otherwise obtained by the processing module 212, rule generating module 214, rule applying module 216, or analysis module 218 may be presented on an interface for display to a user. As another example, visual feedback may be presented on an interface so as to indicate whether rules have been applied to a claims dataset associated with a patient and, if so, whether any of those rules were determined to be a “match.”

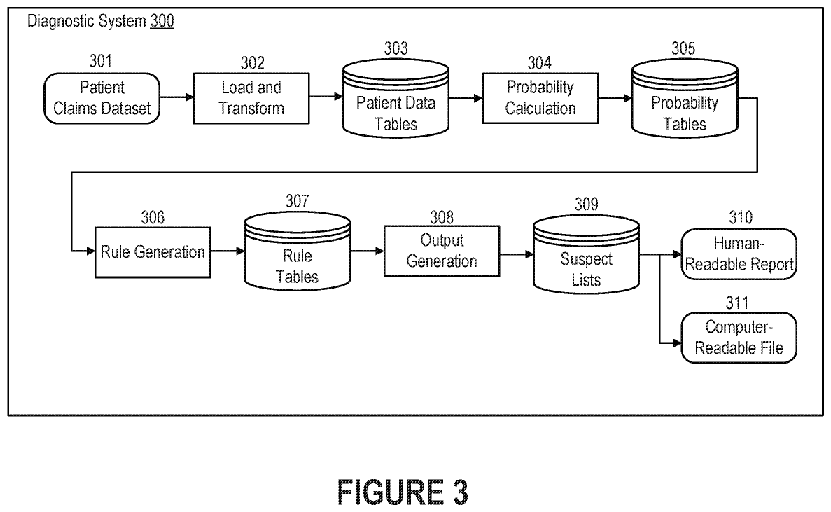

FIG. 3 illustrates how data can be processed as it flows through a diagnostic system 300. The diagnostic system 300 could be the diagnostic system 102 of FIG. 1 or the diagnostic system 210 of FIG. 2. As shown in FIG. 3, the processing flow can be logically segmented into 11 different stages. Note that some stages may not be performed in some embodiments. For example, the claims dataset may not need to be transformed—in which case stage 302 may not be performed—or a human-readable report may not be produced—in which case stage 310 may not be performed.

Initially, claims datasets can be obtained by the diagnostic system (stage 301). Claims datasets may enter the diagnostic system 300 as ANSI files (e.g., ANSI X.12 files) that are formatted in accordance with the American Electronic Data Interchange (“EDI”) standard or in other formats containing similar data elements. Regardless of its form, each claim dataset may include patient claim information or claim reimbursement information. For example, in embodiments where the claims datasets include patient claim information, the claims datasets may be included in electronic files called “837 files.” As another example, in embodiments where the claims datasets include claim reimbursement information, the claims datasets may be included in electronic files called “835 files.” Regardless of form, each claims dataset may include one or more codes that have been assigned to a corresponding patient. These codes may concern diagnoses of the corresponding patient, procedures involving the corresponding patient, or a combination thereof.

Claims datasets could be obtained from providers, insurers, or any combination thereof. In some embodiments, claims datasets may be obtained from providers and insurers and then compared against one another, so as to establish whether a population of patients is accurately represented. Assume, for example, that claims datasets are acquired from several providers who provide general care to a given population of patients (e.g., individuals residing within a given geographical area, such as a state or county). Rules generated based on an analysis of the claims datasets may be erroneous if the claims datasets do not fully represent the patients and diseases of interest. For example, if patients suffering from a given disease (e.g., a cardiovascular disease) are generally referred to a specialized provider for which a claims dataset is not obtained, then rules generated for the given disease may not be appropriate or accurate. By comparing claims datasets acquired from providers and insurers to ensure that a patient population of interest is fairly represented, the diagnostic system 300 can increase its confidence in rules that are generated. Such an approach also ensures that rules generated by the diagnostic system 300 will be more robust as the claims datasets include more “samples” from which to infer relationships.

Thereafter, the diagnostic system 300 can load the claims datasets obtained in the first stage into a first database and then transform the claims datasets into a second database (stage 302). While the first database may be a standard database, the second database may be a proprietary database that is specifically modeled (e.g., with a tabular structure) to facilitate the following stages. Said another way, the diagnostic system 300 may transform the claims datasets between different data structures in order to make the claims datasets easier to handle in subsequent stages. In the second database, the claims datasets can be stored and persisted in a predetermined form to facilitate rule generation (stage 303). Moreover, the claims data may be retained for future iterations of the process shown in FIG. 3, as well as for ad hoc analysis of the process shown in FIG. 3. For example, the claims datasets may be stored in such a manner that the process can be executed when a new claims dataset is acquired by the diagnostic system 300. Upon receiving the new claims dataset, the diagnostic system 300 may re-execute the process using the new and old claims datasets, so as to produce rules based on insights gained through analysis of those combined datasets. Alternatively, the diagnostic system 300 may execute the process using the new claims dataset and then combine any insights learned with those insights learned from prior analysis of the old claims datasets. Previously associated diagnostic groupings may also be stored in the second database if that information is known and available.

In stage 304, the diagnostic system 300 can execute logic (e.g., in the form of SQL program code) that calculates correlation probabilities between datapoints in the claims datasets and the targeted diagnostic grouping for the population under study. This is typically performed for a single provider or insurer. However, this process could be performed in scenarios where the claims datasets are associated with multiple providers or insurers. Moreover, this process could be performed in a more segmented manner, so as to focus on subpopulations (e.g., having a given disease, having a given characteristic, treated by a given professional, etc.) for a single provider or insurer. The datapoints may correspond to treatments prescribed to patients represented in the claims datasets, or the datapoints may correspond to characteristics (e.g., gender, age, ethnicity) of patients represented in the claims datasets. Accordingly, before stage 304 is performed, the diagnostic system 300 may examine the claims datasets in the second database, so as to identify the different datapoints for which correlation probabilities are to be calculated. As an example, the diagnostic system 300 may parse the claims datasets to identify the different datapoints and then batch or group entries that are associated with the same type of datapoint (e.g., the same procedure or same characteristic).

These correlation probabilities can be stored in a data structure (e.g., a probability table). For example, the diagnostic system 300 can store and persist the correlation probabilities computed for the claims datasets in probability tables (stage 305). The probability tables can be maintained for each production run of the calculations, so that changes over time can be recognized and acknowledged. In some embodiments, the diagnostic system 300 is programmed to generate and then store or present a notification in response to a determination that a change in correlation probability exceeds a threshold amount. For example, in the event that the correlation probabilities for a given feature (e.g., procedure or characteristic) decreases by the threshold amount, the diagnostic system 300 may document that the given feature appears to be less correlated with disease than previously thought. Similarly, in the event that the correlation probabilities for a given feature increases by the threshold amount, the diagnostic system 300 may document that the given feature appears to be more correlated with disease than previously thought. Changes in correlation may be useful to professionals that are tasked with determining appropriate courses of action for testing, diagnosing, and treating patients.

In stage 306, the diagnostic system 300 can apply logic (e.g., in the form of SQL program code) to the probability tables to dynamically generate associative rules, applying threshold cutoffs by rule class to establish violation criteria for suspect list inclusion. As a high-level example, a rule might read “X percent of the time, procedure Y is correlated with a diagnostic grouping of A, B, or C.” As a specific example, a rule might read “95 percent of the time, medication apixaban is correlated with a diagnostic grouping atrial fibrillation.” As another specific example, a rule might read “90.8 percent of the time, patients seen by Dr. John Doe have a correlated diagnostic grouping of congestive heart failure.” The rules can be generated in human- and machine-readable format. Note that the threshold cutoffs can be configurable, and therefore could be modified at runtime. Adjusting the threshold cutoffs downward or upward will increase or decrease the level of false positives present in the output, allowing the diagnostic system 300 to be operated at different sensitivity settings based on the needs of the organization.

In stage 307, the diagnostic system 300 can store and persist the rules generated in stage 306. As mentioned above, rules may be created for each population for which probability calculations are performed in stage 304. Tables can then be maintained for each production run of the probability calculations so that changes over time are captured. The rule storage structure may support the manual addition of further logic for refinement.

In stage 308, the diagnostic system 300 again executes logic (e.g., in the form of SQL program code), running a claims dataset associated with a given patient against the rules. The claims dataset (also called “patient dataset” as it pertains to a single patient) can be run against any rule set, including rules generated based on her own claims datasets from the past; rules generated from claims datasets associated with other patients who are associated with the same provider or insurer; rules generated from claims datasets associated with other patients who are associated with different providers or insurers; rules generated from claims datasets associated with other patients who have similar characteristics, have undergone similar procedures, or have been diagnosed with the same disease; or merged rules associated with different providers or insurers. The resulting output can capture which patients have potentially missed diagnostic groupings, which diagnostic grouping is specifically suspected, and which rules triggered the violation.

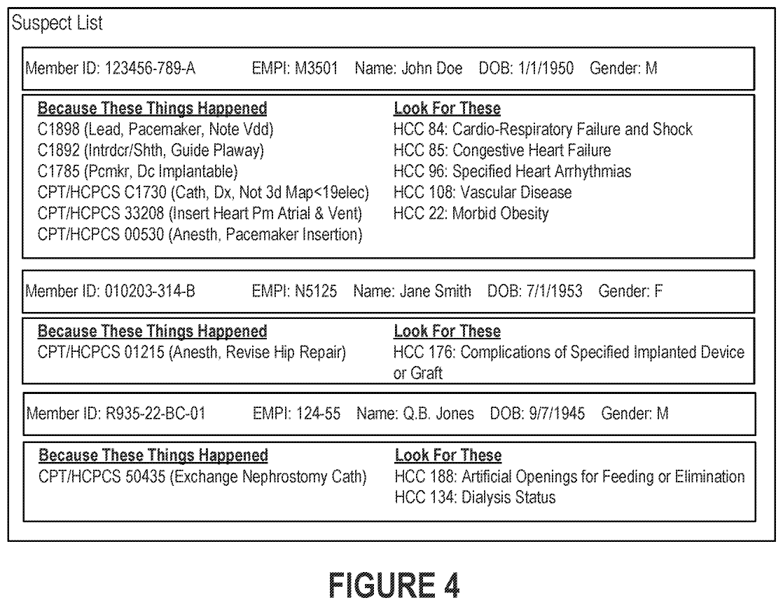

In stage 309, the diagnostic system 300 can store and persist the output from stage 308 in data models that describe the patients with suspected missing diagnostic groupings for each customer. The captured information can include all outputs generated in stage 308, and the captured information can be retained for each production run of the calculations so that changes over time are captured. Outputs can be handled in several different ways as shown in FIG. 3. The outputs may be made available to providers or insurers in human-readable form (stage 310). FIG. 4 includes an example of a suspect list that may be produced by the diagnostic system 300 as output. Additionally or alternatively, the outputs may be made available to providers or insurers in computer-readable form (stage 311), for example, as specified by the providers or insurers at the time of implementation. Data from the data models can be permanently persisted in a local database (stage 309) or transferred to the customer organization via the output formats (stages 310 and 311).

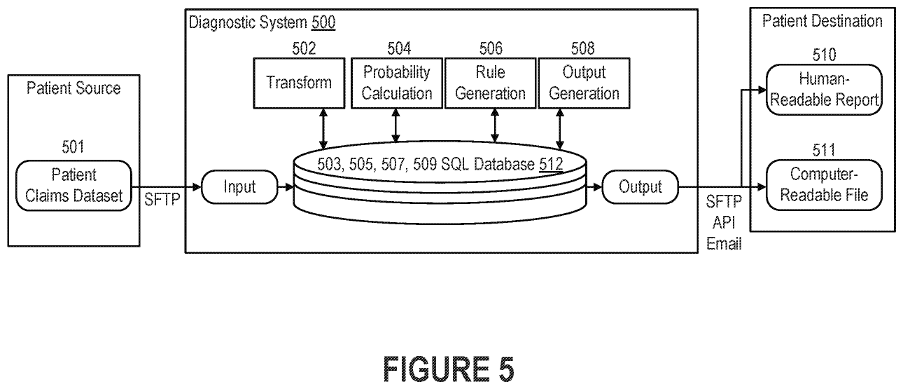

FIG. 5 includes a high-level illustration of an exemplary architecture of a diagnostic system 500. The diagnostic system 400 could be the diagnostic system 102 of FIG. 1, the diagnostic system 210 of FIG. 2, or the diagnostic system 300 of FIG. 3. As mentioned above, the diagnostic system 400 is generally implemented on a cloud-based architecture with principal components of a Structured Query Language (“SQL”) database 512, SQL program components, and corresponding job management scripts. Note that the core design shown in FIG. 5 is largely SQL implementation agnostic, and therefore could be implemented on various SQL services such as PostgreSQL, Amazon Web Services Aurora, Microsoft SQL Server, and Oracle.

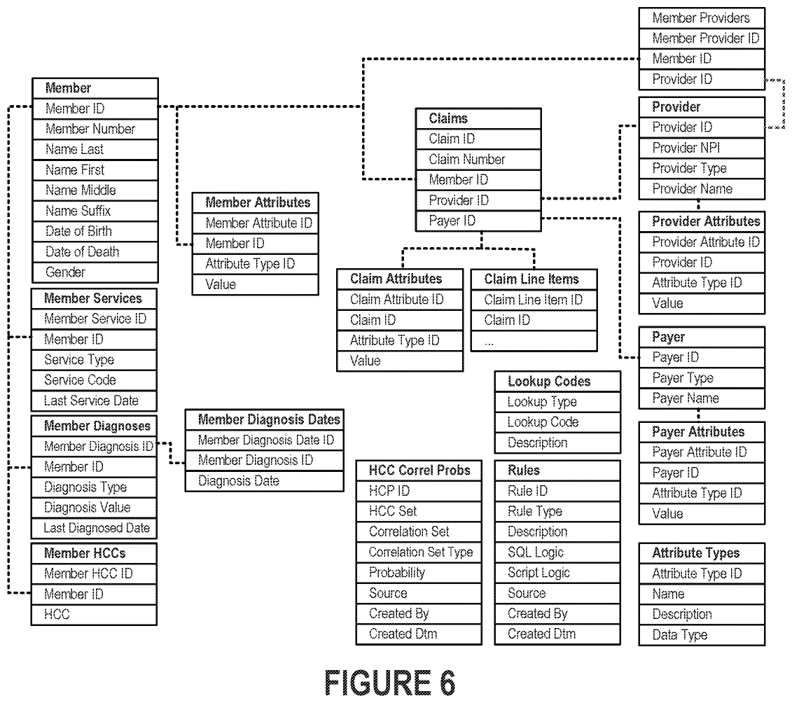

For the purpose of illustration, the reference characters used in FIG. 5 are comparable to those used in FIG. 3. In stage 501, for example, claims datasets that are representative of source data can be obtained from a source external to the diagnostic system 500, such as a storage medium that is managed by, or accessible to, a provider or an insurer. Stage 501 of FIG. 5 is largely comparable to stage 301 of FIG. 3. Note that inbound data is generally received via Secure File Transfer Protocol (“SFTP”) by the diagnostic system 500. Received inbound data can be loaded into the SQL database 512 via a simple, non-transformative database ingest script. Stages 502, 504, 506, and 508 of FIG. 5 may be largely comparable to stages 302, 304, 306, and 308 of FIG. 3. Meanwhile, for stages 503, 505, 507, and 509 of FIG. 5—which may be largely comparable to stages 303, 305, 307, and 309 of FIG. 3—the diagnostic system 500 may utilize proprietary tables maintained in the SQL database 512. An example of a database entity relationship diagram is provided in FIG. 6. Generated output is normally pulled from, or pushed by, the diagnostic platform 500 via a simple database extract script for delivery to a destination external to the diagnostic system 500. The destination may be associated with a provider or an insurer. Output can be conveyed to the provider or the insurer that is representative of the destination via a supported protocol, such as SFTP, application programming interface (“API”), or encrypted email or messaging platform. Output artifacts—shown as stages 510 and 511 of FIG. 5—may be largely comparable to stages 310 and 311 of FIG. 3.

### REPRESENTATIVE EXAMPLES

The following three examples are representative of missed diagnoses that were detected using the diagnostic system described herein. While specific patient information has been redacted, the scenarios represent actual results. The first two examples may also be seen in the example suspect list in FIG. 4. The “Look for These” column in FIG. 4 lists the suspected missed diagnostic groupings. The “Because These Things Happened” column in FIG. 4 lists the rule reasons triggered.

Example 1. Patient Jane Smith received anesthesia for a hip repair revision as detected by a claim containing CPT code 01215. The diagnostic system has a generated rule that states patients receiving that service have an 84.6 percent probability to have been diagnosed with a complication of implanted device (HCC 176). The patient's current records did not reflect that diagnosis. The diagnostic system placed the patient on the suspect list with a clinician note to look for HCC 176.

Example 2. Patient QB Jones received a service to exchange a nephrostomy catheter as detected by a claim containing CPT code 50435. The diagnostic system has a generated rule that states patients receiving that service have an 86.8 percent probability to have been diagnosed with either an artificial opening for feeding or elimination (HCC 188) or are on dialysis (HCC 134). The patient's current records did not reflect that diagnosis. The diagnostic system placed the patient on the suspect list with a clinician note to look for either HCC 188 or HCC 134.

Example 3. Patient Bob Williams was seen by a specific internist located in Utah as detected by a claim containing an office visit charged by this internist. The diagnostic system has a generated rule that states patients seen by this internist have a 90.8 percent probability of diagnosis with congestive heart failure (HCC 85). The patient's current records did not reflect that diagnosis. The diagnostic system placed the patient on the suspect list with a clinician note to look for HCC 85.

Example 4. Patient Mary Nguyen recently filled a prescription for apixaban as detected by a claim containing the NDC code 50090-1436. The diagnostic system has a generated rule that states patients receiving apixaban have a 95.0 percent probability of diagnosis with atrial fibrillation (HCC 96). The patient's current records did not reflect that diagnosis. The diagnostic system placed the patient on the suspect list with a clinician note to look for HCC 96.

### Application and Use Cases

Healthcare employs a variety of diagnostic groupings and taxonomies. These include Hierarchical Condition Categories (“HCCs”), Prescription Drug Hierarchical Condition Categories (“RxHCCs”), Diagnosis-Related Groupings (“DRGs”) such as MS-DRGs and APR-DRGs, Ambulatory Payment Classifications (“APCs”), United Kingdom National Health Service (“NHS”) Healthcare Resource Groups (“HRGs”), and more broadly International Classification of Disease (“ICD”) Categories. The diagnostic platform can detect missed diagnostic groupings represented by any of these groupings and taxonomies through the retrospective, statistical-based approach described herein.

Each of the diagnostic groupings listed above has slightly different applications of the same general capability. For example, detection of missed HCCs can be used to identify reimbursement bonus opportunities for Medicare Advantage plans. Detection of missed HRGs can increase budget accuracy within the UK NHS regional trust system, as well as to other single-payer systems following similar models. Detection of missing ICD Categories can be used to find care gaps across broad clinical populations and applied to population health management.

In summary, the predictive capability of the diagnostic system can be used to close care gaps, enable better treatment, determine patterns of best practice, and ensure that providers, insurers, and risk holders are appropriately reimbursed based on the actual disease states of patients.

### Processing System

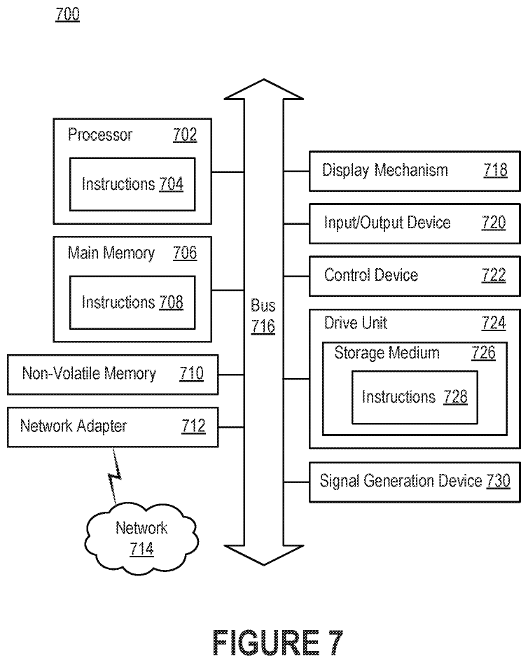

FIG. 7 is a block diagram illustrating an example of a processing system 700 in which at least some operations described herein can be implemented. For example, components of the processing system 700 may be hosted on a computing device on which a diagnostic system is stored and executed.

The processing system 700 may include a processor 702, main memory 706, non-volatile memory 710, network adapter 712, display mechanism 718, input/output device 720, control device 722 (e.g., a keyboard, pointing device, or mechanical input such as a button), drive unit 724 that includes a storage medium 726, or signal generation device 730 that are communicatively connected to a bus 716. The bus 716 is illustrated as an abstraction that represents one or more physical buses and/or point-to-point connections that are connected by appropriate bridges, adapters, or controllers. The bus 716, therefore, can include a system bus, Peripheral Component Interconnect (“PCI”) bus, PCI-Express bus, HyperTransport bus, Industry Standard Architecture (“ISA”) bus, Small Computer System Interface (“SCSI”) bus, Universal Serial Bus (“USB”), Inter-Integrated Circuit (“I²C”) bus, or a bus compliant with IEEE Standard 1394.

While the main memory 706, non-volatile memory 710, and storage medium 726 are shown to be a single medium, the terms “storage medium” and “machine-readable medium” should be taken to include a single medium or multiple media that stores instructions 704, 708, 728. The terms “storage medium” and “machine-readable medium” should also be taken to include any medium that is capable of storing, encoding, or carrying a set of instructions for execution by the processing system 700.

In general, the routines executed to implement the embodiments of the present disclosure may be implemented as part of an operating system or a specific computer programs. Computer programs typically comprise one or more instructions (e.g., instructions 704, 708, 728) set at various times in various memories and storage devices in a computing device. When read and executed by the processor 702, the instructions cause the processing system 700 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile memory and non-volatile memory 710, removable disks, hard disk drives, optical disks (e.g., Compact Disk Read-Only Memory (“CD-ROM”) and Digital Versatile Disks (“DVDs”)), cloud-based storage, and transmission-type media such as digital and analog communication links.

The network adapter 712 enables the processing system 700 to mediate data in a network 714 with an entity that is external to the processing system 700 through any communication protocol supported by the processing system 700 and the external entity. The network adapter 712 can include a network adaptor card, a wireless network interface card, a switch, a protocol converter, a gateway, a bridge, a hub, a receiver, a repeater, or a transceiver that includes a wireless chipset (e.g., enabling communication over Bluetooth or Wi-Fi).

### Remarks

The foregoing description of various embodiments has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the claimed subject matter and its practical applications, thereby enabling those skilled in the relevant art to understand the claimed subject matter, the various embodiments, and the various modifications that are suited to the uses contemplated.

Although the Detailed Description describes certain embodiments, the technology can be practiced in many ways no matter how detailed the Detailed Description appears. Embodiments may vary considerably in their implementation details, while still being encompassed by the present disclosure. Terminology that is used when describing certain embodiments should not be taken to imply that the terminology is being redefined herein to be restricted to any specific characteristics, features, or aspects of the technology with which that terminology is associated. In general, the terms used in the following claims should not be construed to limit the technology to the specific embodiments described in the Detailed Description, unless those terms are explicitly defined herein. Accordingly, the actual scope of the technology encompasses not only the disclosed embodiments, but also all equivalent ways of practicing or implementing the technology.

The language used in the present disclosure has been principally selected for readability and instructional purposes. It may not have been selected to delineate or circumscribe the technology. It is therefore intended that the scope of the present disclosure be limited not by the Detailed Description, but rather by any claims that issue on an application based hereon. Accordingly, the disclosure of embodiments is intended to be illustrative, but not limiting, of the scope of the technology as set forth in the following claims.

What is claimed is:

1. A method for developing a rule that is designed to identify information that is suggestive of a disease upon being applied to claims datasets associated with patients, the method comprising:
acquiring multiple claims datasets from a source,
wherein each of the multiple claims datasets includes codes assigned to a corresponding patient that is known to have the disease;
implementing logic that calculates measures that are representative of correlation between datapoints across the multiple claims datasets and the disease;
generating rules for datapoints, if any, for which the corresponding measures exceed a threshold;
compiling the rules into a set that is associated with the disease; and
storing the set of the rules in a storage medium.

2. The method of claim 1, wherein each measure is representative of a correlation probability metric that indicates the correlation between a corresponding one of the datapoints and the disease, as determined from an analysis of the multiple claims datasets.

3. The method of claim 2, further comprising:
sorting the datapoints into a list that is ordered based on the measures; and
applying, to the list, a filter that removes datapoints for which the corresponding measures do not exceed the threshold.

4. The method of claim 1, further comprising:
causing display of the rules on an interface that is accessible to an individual;
permitting the individual to (i) edit an existing one of the rules, (ii) cancel an existing one of the rules, or (iii) add a new rule through the interface; and
receiving input indicative of a confirmation of the rules by the individual;
wherein said compiling is performed in response to said receiving.

5. The method of claim 1, wherein the codes concern diagnoses of the corresponding patient, procedures involving the corresponding patient, or a combination thereof.

6. The method of claim 1, wherein the source is associated with a healthcare provider that was responsible for providing care to the multiple patients associated with the multiple claims datasets.

7. The method of claim 1, wherein the source is associated with an insurer that was responsible for insuring the multiple patients associated with the multiple claims datasets.

8. The method of claim 1, wherein the datapoints correspond to treatments prescribed to the multiple patients associated with the multiple claims datasets.

9. The method of claim 1, wherein the datapoints correspond to characteristics of the multiple patients associated with the multiple claims datasets.

10. The method of claim 1, wherein the set is associated with the disease by identifying the disease in metadata that is appended to a data structure that is representative of the set and in which the rules are stored.

11. The method of claim 1, wherein the multiple claims datasets are associated with a healthcare professional, and wherein the set is associated with the healthcare professional by identifying the healthcare professional in metadata that is appended to a data structure that is representative of the set and in which the rules are stored.

12. The method of claim 1, wherein the multiple claims datasets are associated with a healthcare provider, and wherein the set is associated with the healthcare provider by identifying the healthcare provider in metadata that is appended to a data structure that is representative of the set and in which the rules are stored.

13. A non-transitory medium with instructions stored thereon that, when executed by a processor of a computing device, cause the computing device to perform operations comprising:
acquiring a claims dataset that includes codes assigned to a patient to which a service has been rendered by a healthcare professional;
identifying a set of rules based on an analysis of the claims dataset or accompanying metadata;
applying, to the claims dataset, the set of rules so as to produce a set of outputs,
wherein each output in the set of outputs indicates whether a corresponding rule in the set of rules was determined to be a match upon being applied to the claims dataset; and
determining an insight into health of the patient based on an analysis of the set of outputs.

14. The non-transitory medium of claim 13, wherein the insight is a positive predicted diagnosis for a disease.

15. The non-transitory medium of claim 14, wherein the operations further comprise:
causing presentation of a notification to the healthcare professional,
wherein the notification recommends that the patient be further examined to render an actual diagnosis for the disease.

16. The non-transitory medium of claim 13, wherein the insight is further based on information related to the individual that is input by the patient, input by the healthcare professional, extracted from a clinical dataset that is associated with the patient, or derived from an electronic health record that is associated with the patient.

17. The non-transitory medium of claim 13, wherein the operations further comprise:
generating a computer-readable file that includes information related to the insight; and
causing transmission of the computer-readable file to a destination.

18. The non-transitory medium of claim 17, wherein the destination is associated with a healthcare provider that employs the healthcare professional.
